(19) United States
(12) Patent Application Publication
(10) Pub. No.: US 2023/0241638 A1
(43) Pub. Date: Aug. 3, 2023

(54) APPLICATOR DEVICE AND APPLICATOR SYSTEM FOR MEDICAMENT CONTAINERS

(71) Applicant: Sanofi, Paris (FR)

(72) Inventor: Bernd Kuhn, Frankfurt (DE)

(21) Appl. No.: 17/927,192

(22) PCT Filed: May 28, 2021
(86) PCT No.: PCT/EP2021/064321

§ 371 Date: Nov. 22, 2022

(30) Foreign Application Priority Data
May 29, 2020 (EP) 20315278.0

(51) Int. Cl. B05B 13/06; A61M 5/31; B05B 1/20; B05B 15/70

(52) U.S. Cl. CPC B05B 13/0627; A61M 5/3129; B05B 1/20; B05B 15/70; B05B 13/0618; A61M 2005/3131

(57) ABSTRACT

The present disclosure relates to an applicator device for depositing a lubricant onto an inside surface of a barrel of a medicament container. The applicator device includes a sprayer defining a longitudinal direction and sized for insertion into an interior of the barrel along the longitudinal direction. The sprayer is operable to eject the lubricant. A centering element is connected to the sprayer and operably engageable with the barrel in a predefined position or orientation to align the sprayer relative to the barrel.

### CROSS REFERENCE TO RELATED APPLICATIONS

The present application is the national stage entry of International Patent Application No. PCT/EP2021/062796, filed on May 14, 2021, and claims priority to Application No. EP 20315241.8, filed on May 15, 2020, the disclosures of which are incorporated herein by reference.

### TECHNICAL FIELD

The present disclosure relates to an applicator device, to an applicator system as well as to a method of depositing a lubricant onto an inside surface of a barrel of a medicament container. The disclosure particularly relates to lubricant application to medicament containers equipped or provided with a piston, stopper or plunger movably arranged inside the barrel. The disclosure particularly relates to cartridges for use with drug injection devices. It may also relate to syringes as well as to vials or capsules filled with a liquid medicament.

Particularly, the disclosure relates to the coating of an inside surface of a medicament container with a lubricant, wherein the medicament container is configured or operable for dispensing of a liquid medicament by way of expelling the liquid medicament from the barrel of the medicament container.

### BACKGROUND

Medicament containers, such as cartridges or prefilled syringes either for manual use or for use in medical devices, such as a pen-type injectors, wearable pumps or auto-injectors should provide reliable performance for an accurate dose delivery. Thus, reproducible force profiles enabling to move a piston or plunger stopper inside a barrel of such containers are of particular importance.

To achieve low and constant friction for a piston or stopper configured for a sliding displacement inside the barrel of the medicament container the inside of the barrel can be in principle provided with a thin layer of a lubricant. In industrial mass manufacturing processes for providing a rather large quantity of medicament containers there may occur an incomplete or inhomogeneous distribution of the lubricant inside the medicament container.

This may eventually lead to an increased variability of frictional forces or force profiles for moving the piston or stopper relative to the barrel of the medicament container. This may have a particular impact for automated injection devices or delivery systems providing only a predetermined and, for example, limited power source for moving the piston or stopper relative to the barrel.

It is therefore desirable to improve an inside surface coating of such medicament containers with a lubricant. The lubricant should be applied homogeneously on the inside surface of such barrels or medicament containers. It is further desirable to provide a precise, cost efficient and highly reliable coating of the barrel with a lubricant, in particular for a mass manufacturing of medicament containers. Hence, a desirable improvement for applying the lubricant onto the inside surface of the barrel should be suitable for implementation in a rather cost-efficient mass manufacturing process, which is, for example, fully or semi-automated.

### SUMMARY

In one aspect there is provided an applicator device for depositing a lubricant onto an inside surface of a barrel of a medicament container. The applicator device comprises a sprayer defining a longitudinal direction. The sprayer is sized for insertion into an interior of the barrel along the longitudinal direction. The sprayer is operable to eject the lubricant. The applicator device further comprises a centering element connected to the sprayer. The centering element is operably engageable with the barrel in a predefined position or orientation to align the sprayer relative to the barrel. The centering element is particularly mechanically engageable with the barrel in a releasable manner.

At least during the process of applying the lubricant to the inside surface of the barrel the centering element may be in contact with the barrel. The sprayer may be fixable relative to the centering element at least with regard to one degree of movement or degree of freedom. With some examples, the sprayer is even fixable relative to the centering element with regard to at least two degrees of movement or to degrees of freedom. With some examples, the sprayer may be even fixed to the centering element with regard to three degrees of movement.

Due to the connection between the sprayer and the centering element and the ability of the centering element to mechanically and/or operably engage with the barrel the sprayer can be kept in a well-defined position, orientation and/or alignment relative to the barrel. In this way, a precise position, alignment or orientation of the sprayer relative to the barrel can be provided. Such a well-defined positioning, alignment or orientation of the sprayer relative to the barrel is of particular benefit in order to apply the lubricant to the inside surface of the barrel in a rather homogeneous and well-defined way.

With some examples, the centering element may be sized to become inserted into the interior of the barrel. Here, the centering element may be located longitudinally offset from a nozzle of the sprayer. The centering element may be located close to a nozzle of the sprayer so as to define a well-defined position or distance of the sprayer to the inside surface of the barrel.

The centering element particularly serves to demobilize the sprayer relative to the barrel of the medicament container with regard to a transverse plane or transverse direction, wherein the transverse plane or transverse direction extends substantially perpendicular to the longitudinal direction as defined by the sprayer or barrel. The sprayer may comprise an elongated shape. When the barrel is also of elongated shape, e.g., of cylindrical shape, the sprayer may be configured for a coaxial alignment with the medicament container as it is inserted in longitudinal direction into the container. The centering element may then serve to define or to maintain a transverse or radial distance between the sprayer and the inside surface of the barrel at least during application or depositing of the lubricant.

Of course, the sprayer is connectable or is connected to a lubricant feeding system. The lubricant feeding system is typically equipped with a reservoir for the lubricant. The lubricant feeding system typically comprises a pump and at least some kind of a hose or pipe way of which the sprayer is in fluid communication with the pump or reservoir. By activating the pump, the sprayer can be provided with the lubricant. Typically, the sprayer comprises at least one nozzle to atomize the lubricant and/or to homogeneously moisture or sprinkle the inside surface with the atomized lubricant.

According to another example the sprayer is movable relative to the centering element with regard to the longitudinal direction. In this way the centering element may remain stationary relative to the barrel as the sprayer is moved relative to the centering element and relative to the barrel in the course of applying or depositing the lubricant to the inside surface of the barrel. In this way, the centering element may, for example, engage with a longitudinal end of the barrel while the sprayer is inserted into the interior of the barrel in longitudinal direction. Here, the sprayer is allowed to move at least in longitudinal direction relative to the barrel, thereby providing a homogeneous application of the lubricant along the longitudinal direction or longitudinal elongation of the barrel or medicament container.

Even though the sprayer is movable relative to the centering element, the centering element may serve to demobilize the sprayer with regards to at least one degree of freedom, preferably with regard to two degrees of freedom extending substantially perpendicular to the longitudinal direction. Here, with a cylindrical shape of the barrel, the sprayer may be movable along the axial or longitudinal direction relative to the centering element, while it is locked in radial direction to the centering element.

Keeping the sprayer movable relative to the centering element particularly allows to engage the centering element, for example, with a proximal end of the barrel. In this way it can be avoided that the centering element enters the interior of the barrel. In this way, an interaction or contact of the interior of the barrel with external components, such as the centering element, can be reduced to a minimum.

Through the longitudinal movability of the sprayer relative to the centering element, the centering element may mechanically engage with an outside surface or outside portion of the barrel, while the sprayer is inserted into the interior of the barrel while keeping the sprayer in a predefined radial or transverse distance to the inside surface of the barrel. With the help of the centering element, the sprayer can be inserted into the interior of the barrel without making contact to a sidewall of the barrel or to any other component or portion of the barrel. The sprayer may remain entirely contactless with regard to the barrel. Contactless operation shall ensure that the lubricant spray reaches the barrel surface as a spray or mist. If the sprayer gets in contact to the barrel surface, the lubricant spray may condense and may flow down along the side wall of the barrel, thus leading to inhomogeneous lubricant distribution.

According to another example, the applicator device comprises a sliding guide by way of which the sprayer is slidably displaceable relative to the centering element with regard to the longitudinal direction. The sliding guide may be constituted by the mutual mechanical interaction of the sprayer and the centering element. With some examples the sliding guide may be provided by the centering element. With other examples the sliding guide may be provided by the sprayer. With every conceivable implementation of the sliding guide the sprayer is slidably displaceable relative to the centering elements with regard to the longitudinal direction.

Concurrently, the sprayer may be fixed and immobilized to the centering element with regard to a transverse direction or with regard to the transverse plane extending substantially perpendicular to the longitudinal direction. If the sprayer or the medicament container comprise a somewhat cylindrical geometry, the longitudinal direction may coincide with the cylinder long axis. The transverse direction may then be described by the radial direction with regard to the long axis of the respective cylindrical geometry.

The sliding guide may further enable a stepless and/or continuous movement of the sprayer relative to the centering element. When the centering element is immobilized with regard to the longitudinal direction relative to the barrel the sliding movement of the sprayer relative to the centering element correspondingly transfers into a respective sliding movement of the sprayer relative to the barrel. In this way, the sprayer can be moved steplessly and rather continuously relative to the barrel. For instance, the sprayer can be moved with a constant velocity relative to the barrel while a constant flow of lubricant is ejected by the sprayer onto the inside surface of the barrel. A constant volumetric flow of the lubricant ejected by the sprayer combined with a constant velocity of moving the sprayer in longitudinal direction relative to the barrel may provide a rather homogeneous coating of the inside surface with the lubricant.

Generally, the sliding guide can be implemented in numerous different ways. At least one of the sprayer and the centering element comprises a longitudinally extending guide or rail whereas the other one of the sprayer and the centering element is movable along this longitudinal guide or rail. With some examples, it is the sprayer that has a longitudinal guide or rail which is in longitudinal sliding engagement with the centering element. With other examples, it may be the centering element that comprises a longitudinally extending guide or rail in sliding engagement with the sprayer.

According to another example the centering element comprises a central hub section with a through opening. The sprayer comprises an elongated shaft section slidably supported in the through opening. The shaft section comprises a rather constant diameter or cross-section as seen in longitudinal direction. Typically, an outside diameter or outside cross-section of the elongated shaft section corresponds or is complementary shaped to a respective inside diameter or insight cross-section of the central hub section. In this way, the elongated shaft section of the sprayer is in longitudinal sliding engagement with the through opening of the central hub section.

Since the elongated shaft section of the sprayer is enclosed by the through opening, the elongated shaft section and hence the entire sprayer may be confined in the transverse plane or with regard to the radial direction by the through opening. The elongated shaft section extending in longitudinal direction through the through opening of the central hub section provides a longitudinal sliding motion of the sprayer relative to the centering elements. At the same time the sprayer can be immobilized or fixed to the through opening and to the central hub section at least with regard to the radial direction or to the transverse plane. In this way, for being longitudinally slidably engaged with the central hub section and the centering element the sprayer can be kept in a well-defined radial or transverse distance to the inside surface of the barrel.

Typically, the centering element is configured and operable to engage with the barrel of the medicament container in a well-defined configuration with regards to the radial direction or transverse direction. In this way, a well-defined longitudinal alignment and orientation as well as longitudinal and transverse positioning of the sprayer relative to the barrel can be provided.

According to a further example, the centering element comprises an outer sidewall coaxial with the central hub section and enclosing the central hub section. With the outer sidewall, the transverse extension or transverse geometry of the centering element can be enlarged compared to the central hub section. In this way, the centering element may comprise a diameter or cross-section exceeding a respective diameter or cross-section of the barrel of the medicament container. Hence, the centering element back be configured or may be operable for longitudinal abutment with a longitudinal end, e.g. with the proximal end of the barrel of the medicament container.

The outer sidewall may comprise or may form a flange section extending radially outwardly from the central hub section. The outer sidewall may be provided by such a flange section of the centering element. A distal side of the flange section of the centering element may be configured to engage and/or to abut in longitudinal direction with a proximal end of the barrel. In this way, the centering element may be fixable relative to the barrel at least with regard to the longitudinal direction. Moreover, at least one of the outer sidewall, the central hub section and/or an optional flange extending between the outer sidewall and the central hub section may be shaped to engage with the barrel in one well-defined position or orientation as seen in the transverse or radial direction. In this way, the centering element may be radially or transversely centered with regard to the transverse direction or radial extent of the barrel.

According to another example, the central hub section and the outer side wall are interconnected by at least three connecting wall sections extending radially between the central part section and the outer side wall. Typically, the diameter or cross-section of the central hub section is smaller than the diameter or cross-section of the sidewall of the barrel. A diameter or cross-section of the outer sidewall of the centering element is typically larger than the outside diameter or outside cross-section of the medicament container. The at least three connecting wall sections typically comprise a slanted shape as seen in the longitudinal and radial direction. Typically, and when the centering element is configured for longitudinal engagement or abutment with the proximal end of the barrel, the at least three connecting wall sections might be triangularly shaped as seen in a plane defined by the longitudinal and the radial direction.

Towards the proximal direction, the radial extent of the connecting wall sections may increase. Towards the distal direction, the radial extent of the connecting wall sections may decrease. Typically, the at least three connecting wall sections are equidistantly distributed along the circumference of the central hub section. In this way, a kind of a self-centering of the centering element relative to the barrel can be provided with regard to the radial or transverse direction as the central hub section is brought in longitudinal or axial engagement with the barrel. Here, the centering element, in particular its central hub section, may be at least partially inserted into the proximal open end of the barrel of the medicament container.

The at least three connecting wall sections are of somewhat identical shape. They may be arranged at an angular distance of about 120° around the circumference of the central hub section. The distally facing edge of the connecting wall section may be slanted. Here, an inside or radial inner end of the edge of the connecting wall section is located distally offset from the radial or transverse outer end of a distally facing edge of the connecting wall section. The distally facing edge of the connecting wall sections may comprise a rather straight or curved shape. It may comprise a concave shape providing a rather smooth transverse self-centering of the centering element as it is at least partially inserted in longitudinal direction into the proximal end of the barrel of the medicament container.

Instead of at least three connecting wall sections there may be provided a plurality of connecting wall sections, e.g., 4, 5, 6, 8, 10 or 12 to 15 connecting wall sections. Further alternatively, and instead of the at least three connecting wall sections a radial flange interconnecting the outer sidewall and the central hub section may be somewhat cone shaped and may comprise a rather closed surface.

However, the at least three connecting wall sections might be beneficial in that they do not close off the interior of the medicament container when in longitudinal abutment with the proximal end of the barrel. In this way, and while the centering elements might be in axial abutment or mechanical engagement with a longitudinal end of the barrel, excess lubricant may drain out of the barrel substantially unhinderedly.

According to a further example, the sprayer is movable in a longitudinal distal direction relative to the centering element against a restoring force of a restoring element engaged with the sprayer and engaged with the centering element. With some examples the restoring element comprises a spring element. The spring element may comprise a helically shaped compression spring. One longitudinal end of the spring or restoring element may be engaged with the centering element. An oppositely located longitudinal end of the spring or restoring element may be engaged with the sprayer. With some examples, a distal end of the restoring element or spring is in longitudinal abutment with a proximally facing abutment of the centering element. A proximal end of the spring is in longitudinal abutment with a distally facing abutment of the sprayer.

In this way the sprayer is displaceable in distal longitudinal direction relative to the centering element, thereby transferring, e.g., compressing, the restoring element against the restoring force into a biased configuration. With such a restoring element, a twofold function can be provided. First of all, and when the sprayer is moved in distal direction in unison with the centering element relative to the barrel, the longitudinal distally directed movement may lead to a longitudinal engagement of the centering element with the barrel. When the centering element is in an abutment configuration with the barrel, and when the sprayer is moved further in distal direction, it may start to slide in longitudinal direction relative to the centering element. In this way, the sprayer may enter and may be inserted into the interior of the barrel while the centering elements rests against, for example, a proximal end face of the barrel.

This insert movement of the sprayer into the barrel and hence the movement of the sprayer relative to the centering element is accompanied by a biasing of the restoring element. A withdrawal of the sprayer out of the interior of the barrel may be thus provided and supported by the restoring element. Hence, the restoring element may serve to remove the sprayer from the interior of the barrel.

Moreover, as long as the sprayer is inserted into the barrel, the restoring element applies a respective pressure in distal direction onto the centering element, thereby keeping the centering element in axial or longitudinal engagement with the barrel of the medicament container. As long as the centering element is biased in the longitudinal direction against, for example, the proximal end of the barrel it is remained in a well-defined self-centered radial or transverse position relative to the barrel and serves to center the sprayer with regard to the lateral or radial direction.

Use of the restoring element may be also beneficial in a mass manufacturing process. With some examples, the centering element may be supported on the sprayer. It may be mounted on the sprayer and may be movable in longitudinal direction relative to the sprayer against the action of the restoring element under the action of the restoring element. Insofar, a separate handling for the centering element may not be required. It may be located and mounted on, for example, a distal end of the sprayer and may be movable against the action of the restoring element towards a proximal end of the sprayer in the course of inserting the sprayer into the interior of the barrel from the open proximal end of the barrel.

Furthermore, the restoring element may provide a kind of a suspension and shock absorption, namely when the centering elements gets in longitudinal abutment with a proximal end of the barrel of the medicament container. This particularly applies when the sprayer with the centering element mounted thereon is subject to a movement relative to the barrel.

According to a further example, the centering element comprises at least one of an abutment to abut in longitudinal direction against a proximal end of the barrel, and a receptacle to receive the proximal end of the barrel. Both, the abutment and the receptacle may provide a self-centering of the centering element when bringing the centering element in longitudinal abutment with a proximal end of the barrel. Both, the abutment and the receptacle, may provide a well-defined longitudinal abutment of the centering element to the proximal end of the barrel. In this way, a well-defined mechanical engagement between the barrel and the centering element and hence the applicator device can be provided.

According to a further example, at least one of the abutment and the receptacle comprises a slanted section extending at a predefined angle relative to the longitudinal direction. The slanted section is configured to induce a lateral movement of the centering element relative to the medicament container when the proximal end of the container gets in longitudinal abutment with at least one of the abutment and the receptacle. For this, the slanted section and/or the receptacle might be slanted in the plane defined by the longitudinal and the transverse or radial direction. The receptacle may be slanted and may engage with an outside facing sidewall of the barrel. With some examples, the at least three connecting wall sections form, confine or constitute at least one of the abutment and the receptacle. They may contribute to the abutment and/or to the receptacle.

The proximal end of the barrel is typically of cylindrical or circular shape. The slanted or cone-shaped section of the receptacle or of the abutment provides a self-centering of the centering element when getting in axial or longitudinal abutment or engagement with the proximal end of the barrel. The slanted section of at least one of abutment and the receptacle may be formed radially between the central hub section and the outer sidewall. The receptacle may be radially or transversally confined by the outer sidewall. The same may apply to the abutment of the centering element.

With some examples, an inside facing surface of the outer sidewall may be slanted and may belong to or may constitute the receptacle. The abutment may be provided radially or transversely inwardly from the sidewall of the barrel. It may be also slanted or cone-shaped so as to engage with an inside facing portion of the barrel of the medicament container.

According to another example, the sprayer comprises a longitudinal fluid guiding bore enclosed by a longitudinally extending side wall. The sprayer further comprises a nozzle at or near a distal end of the longitudinally extending side wall. The nozzle is in fluid communication with the fluid guiding bore. The nozzle further comprises at least one orifice extending radially or transversely through the side wall. The longitudinal direction of the sprayer typically coincides with the elongation of the longitudinally extending sidewall. The sidewall of the sprayer may be cylindrically shaped. The cylindrical sidewall of the sprayer is hollow and comprises the fluid guiding bore. The nozzle at or near the distal end of the sidewall of the sprayer serves to atomize the lubricant as the lubricant is forced through the fluid guiding bore towards the nozzle. The nozzle typically extends radially outwardly so as to apply and/or to deposit the lubricant radially outwardly onto the inside surface of the barrel when the sprayer is located in the interior of the barrel.

With some examples the nozzle is implemented as a so-called single-component nozzle. With a single-component nozzle, the lubricant is subject to atomization or nebulization based on the pressure-drop across the nozzle, i.e. between the liquid lubricant in a feeding line or fluid guiding bore upstream of the nozzle and a region outside or downstream of the nozzle. This pressure drop may substantially provide the energy for an atomization of the lubricant.

With other examples the nozzle is implemented as a two-component nozzle being in fluid communication with a lubricant feeding line and with a pressurized gas. Here, the lubricant is a first component, i.e. a liquid component and the pressurized gas is a second component, i.e. a gaseous component. The second component serves to atomize the first component as the first and the second components are ejected via the nozzle concurrently.

Typically, the longitudinally extending sidewall coincides with the shaft of the sprayer, which is slidably supported in the through opening of the central hub section of the centering element. In other words, the longitudinally extending sidewall of the sprayer forms or constitutes the elongated shaft of the sprayer. Accordingly, the longitudinally extending sidewall is in longitudinal sliding engagement with the centering element.

The sprayer may comprise not only one but several nozzles. These nozzles typically extend in different or opposite direction along the outer circumference of the cylindrically-shaped sidewall or shaft. In this way, a rather homogeneous application of the lubricant to the inside surface of the barrel of the medicament container can be provided.

According to a further example, the nozzle comprises at least a first orifice and a second orifice. The first orifice is located offset from the second orifice as seen in circumferential direction of the longitudinally extending side wall or shaft of the sprayer. In this way, diametrically oppositely located sections in the surrounding of the shaft or longitudinally extending sidewall of the sprayer can be supplied or provided with atomized lubricant.

The size of the first orifice and/or of the second orifice may be comparatively large as seen in circumferential or tangential direction of the cylindrically-shaped sidewall or shaft of the sprayer. In this way, a spatially homogeneous deposition of lubricant to the inside surface of the barrel can be provided in general.

According to a further example, the nozzle comprises at least a first orifice and a third orifice. The first orifice is located offset from the third orifice as seen in longitudinal or axial direction and as seen in circumferential direction of the longitudinally extending sidewall or shaft. Moreover, as seen in tangential or circumferential direction the first orifice and the third orifice may at least slightly overlap. In this way, a spatially homogeneous deposition of atomized lubricant on the inside surface of the barrel can be provided.

With some examples, the nozzle comprises a first orifice and a second orifice diametrically opposite to each other. The nozzle may comprise a third orifice and a fourth orifice. The third orifice and the fourth orifice being also arranged diametrically opposite to each other. As seen in circumferential or tangential direction, the pair of first and second orifices and the pair of third and fourth orifices may be arranged at a circumferential offset. As seen in tangential direction the third orifice may be located in the intermediate space between the first and second orifices. Also, the fourth orifice may be arranged circumferentially between the first and the second orifice. In this way, an inevitable shadow section inevitably provided between the first and second orifices may be covered by at least one of the third and fourth orifice of the nozzle.

With some examples, a virtual interconnecting line extending from the first orifice to the second orifice may be oriented at about 90° relative to a second virtual interconnecting line interconnecting the third orifice and the fourth orifice.

In effect and as seen in circumferential or tangential direction, the arrangement of first, second and optional third and fourth orifice provides a rather spatially homogeneous atomizing of the lubricant in the vicinity of the sprayer. Typically, the nozzle provides a 360° distribution of atomized lubricant at or near the distal end of the shaft section of the sprayer.

According to a further example, the nozzle comprises a nozzle grid. The nozzle grid may comprise a sintered filter or sintered sieve structure or a respective sintered metal structure. The nozzle grid may comprise a mesh of sintered metal providing a large number of rather tiny orifices to provide atomizing of the lubricant as it is forced through the fluid guiding bore.

The orifices as mentioned above as well as the nozzle grid are provided in the longitudinally extending sidewall of the nozzle. A distal end of the nozzle may be implemented as a dead end and may close off the longitudinal fluid guiding bore so as to prevent expelling or ejecting of the lubricant in distal direction. With the orifices and/or the nozzle grid provided in a distal section of the longitudinally extending sidewall or shaft, a deposition of the lubricant on an inside surface of the sidewall of the barrel can be provided while keeping, for example, a radially inwardly extending shoulder portion of the barrel or a distal end of an inside surface of the barrel void of the lubricant.

According to a further aspect, the disclosure also relates to an applicator system for depositing a lubricant onto an inside surface of a barrel of a medicament container. The applicator system comprises at least one applicator device as described above. The applicator system further comprises a lubricant feeding system in flow connection with a sprayer of the applicator device as described above. The applicator system further comprises at least one electromechanical actuator. The electromechanical actuator being operable to move the applicator device relative to the medicament container. Moreover, the applicator system comprises a controller connected to the lubricant feeding system and connected to the at least one electromechanical actuator. The controller is operable and/or configured to control a relative movement of the applicator device relative to the medicament container and to control ejection of the lubricant.

The applicator system is particularly configured to automatically conduct deposition of the lubricant onto the inside surface of the barrel. The controller is particularly configured to induce a relative movement of the applicator device relative to the barrel so as to insert the sprayer of the applicator device into the interior of the barrel. Once the sprayer has been appropriately inserted into the interior of the barrel the control may induce or control dispensing or injection of the lubricant. During dispensing or injecting of the lubricant the sprayer may be withdrawn from the interior of the barrel at a predefined velocity.

The applicator system is particularly configured for a mass manufacturing of medicament containers. The applicator device may comprise numerous sprayers, each of which provided with a centering element. The applicator system may further comprise a mount with numerous receptacles to retain a number of barrels. Accordingly, the applicator system may also comprise a mount for retaining numerous applicator devices as described above. For instance, the applicator device may comprise a mount comprising up to 10, up to 12 or even up to 15 or 20 individual receptacles for a medicament container. Correspondingly, the applicator system may comprise a mount for a corresponding number of sprayers and centering elements.

The at least one electromechanical actuator is mechanically engaged or connected to at least one of the mount for the applicator device(s) and the mount for the medicament container(s). In this way and with a single electromechanical actuator, a large number of applicator devices and medicament containers can be moved relative to each other simultaneously. In this way, a rather large number of medicament containers can be provided with the lubricant simultaneously. Since each sprayer of a set of numerous sprayers is provided with a separate centering element, a well-defined position, alignment or orientation of each sprayer relative to the barrel of the respective medicament container can be provided.

According to another aspect, the present disclosure relates to a method of depositing a lubricant on an inside surface of a barrel of a medicament container. The method comprises the steps of providing at least one medicament container. In a further step, a sprayer of the applicator device as described above is inserted into the medicament container. During or in the course of inserting the sprayer into the medicament container, the centering element of each sprayer is used to align and/or to radially center the sprayer relative to the barrel of the medicament container. Centering is typically conducted along a transverse direction or with regard to the transverse plane extending substantially perpendicular to the elongation of the sprayer or the elongation of the barrel of the medicament container. In a further step, an amount of the lubricant is ejected onto the inside surface of the barrel by using the sprayer.

Generally, the method of depositing a lubricant on an inside surface of a barrel is implemented by making use of an applicator device and/or of an applicator system as described above. Insofar all features, effects and benefits as described above in connection with the applicator device and the applicator system equally apply to the method of depositing a lubricant on an inside surface of the barrel; and vice versa.

According to another aspect, there is provided a sprayer for an applicator device as described above. The sprayer comprises an elongated shaft or a longitudinally extending side wall. The sprayer further comprises a longitudinal fluid guiding bore extending in longitudinal direction through the longitudinally extending side wall or shaft. Typically, at a distal end or near a distal end, which is typically closed off, the shaft or the longitudinally extending sidewall comprises a nozzle in fluid communication with the fluid guiding bore. The nozzle comprises at least one orifice extending radially through the sidewall.

Typically and according to a further example, the nozzle of the sprayer comprises a first orifice and a second orifice. The first orifice is located offset from the second orifice as seen in circumferential direction of the longitudinally extending sidewall as seen in circumferential direction of the substantially cylindrically shaped shaft of the sprayer. The first orifice and the second orifice may be oriented diametrically opposite to each other. As seen in circumferential direction the first orifice and/or the second orifice may comprise an opening angle of at least 15°, of at least 20°, of at least 30°, of at least 45°, of at least 60° or of at least 90°. A comparatively large opening angle as seen in circumferential direction provides a rather widespread atomizing of the lubricant when forced through the fluid guiding bore and injected via the nozzle.

With a further example the shaft or the longitudinally extending side wall comprises a third orifice located offset from the first orifice as seen in longitudinal direction and as seen in circumferential direction of the elongated shaft. The third orifice may be oriented and/or arranged circumferentially offset from the first orifice. The opening range or opening angle of the third orifice may adjoin or may even overlap with the opening angle of the first orifice as seen in a longitudinal projection. In this way, the third orifice may be located circumferentially between the first orifice and the second orifice. A blind area or shadow area inevitably provided between the first and the second orifice may be covered by the third orifice.

The sprayer, typically it's elongated shaft or longitudinally extending sidewall, may be also provided with a fourth orifice located diametrically opposite to the third orifice. Typically, the first and the second orifices may be located at a common first transverse plane that is longitudinally offset from a second transverse plane. The third and the fourth orifice are located in or overlap with the second transverse plane.

With the first, the second, the third and the fourth orifice, a spatially homogeneous atomizing of the lubricant in the surrounding of the shaft or in the surrounding of the longitudinally extending sidewall can be provided, e.g., around 360°.

With another example, the sprayer comprises a base section and the elongated shaft section. Here, the shaft section may be rotatable relative to the base section of the sprayer. Alternatively, and when the shaft section and the base section are mutually fixed, the base section together with the shaft section may be subject to a rotation with the longitudinal axis as an axis of rotation at least during an ejecting of the lubricant. With a rotational support for the sprayer, the sprayer may comprise only one nozzle with a single orifice.

During injection of the lubricant through the orifice, the orifice and hence the longitudinally extending side wall or shaft of the sprayer may be subject to a rotational motion relative to the barrel.

According to a further aspect, the disclosure also relates to a medicament container coated or provided with a lubricant applied to the container by making use of an applicator device, an applicator system and/or by executing the method as described above. The medicament container comprises a barrel provided with the lubricant on an inside surface of the barrel. The medicament container is filled with a liquid medicament and is typically sealed in proximal direction with a movable piston or stopper.

Generally, the scope of the present disclosure is defined by the content of the claims. The applicator device, the applicator system and the respective method of depositing the lubricant are generally not limited to specific embodiments or examples, but comprise any combination of elements of different embodiments or examples. Insofar, the present disclosure covers any combination of claims and any technically feasible combination of the features disclosed in connection with different examples or embodiments.

In the present context, the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel, configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 3 years. Storage may occur at room temperature (e.g., about 20° C.), or refrigerated temperatures (e.g., from about 2-8° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2019, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, examples of an applicator device and an applicator system for depositing a lubricant onto an inside surface of the barrel of a medicament container will be described in greater detail by making reference to the drawings, in which:

FIG. 1 shows an example of a pen-type injection device configured to be equipped with a medicament container,

FIG. 2 shows numerous components of the injection device of FIG. 1,

FIG. 3 is a side view of an applicator device before inserting a sprayer into the interior of a medicament container,

FIG. 4 is a side view in accordance to FIG. 3, wherein the sprayer is inside the interior of the barrel and wherein an ejection of the lubricant to an inside surface of the barrel has started,

FIG. 5 shows the applicator device after removal of the sprayer from the interior of the barrel,

FIG. 6 shows a top view of an isolated centering element of the applicator device,

FIG. 7 shows a detailed side view of the mutual assembly of a sprayer and a centering element of an applicator device,

FIG. 8 is a perspective illustration of a sprayer,

FIG. 9 shows a distal end of the sprayer of FIG. 8 in an enlarged view,

FIG. 10 shows another example of a distal end of a sprayer having a grid or sinter-metal nozzle,

FIG. 11 is a cross-section along A-A of FIG. 9,

FIG. 12 is a cross-section through B-B of FIG. 9,

FIG. 13 shows a block diagram of an applicator system including an applicator device,

FIG. 14 shows a part of the applicator system provided with a mount for a number of medicament containers and further provided with a mount for a number of applicator devices, and

FIG. 15 shows a flowchart of a method of depositing a lubricant on an inside surface of a barrel.

### DETAILED DESCRIPTION

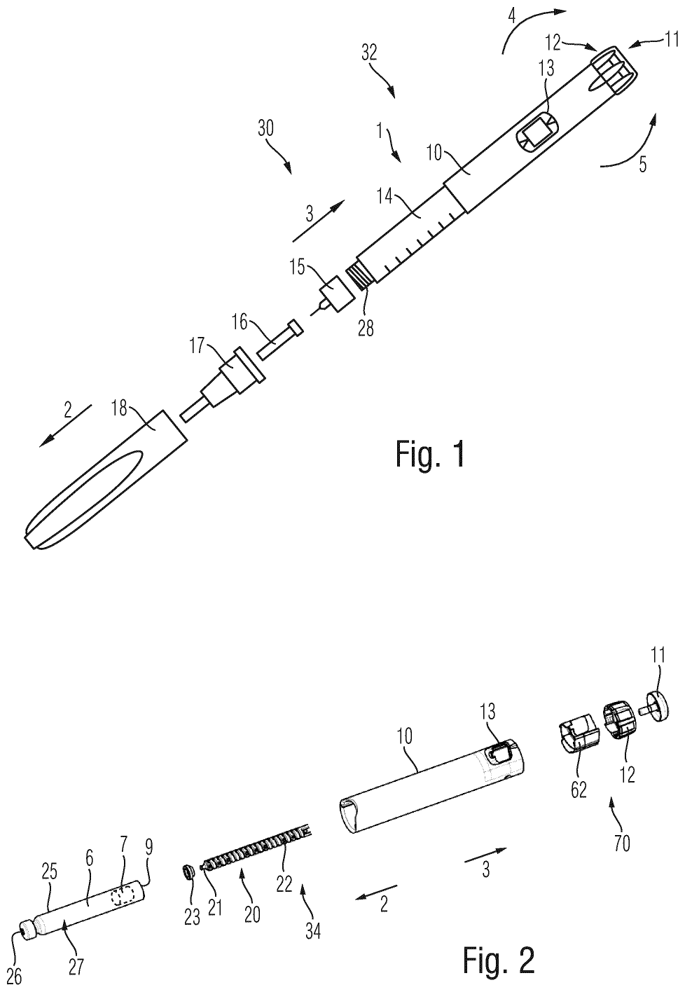

One example of a drug delivery device 1 for administering of a dose of a medicament 27 is illustrated in FIGS. 1 and 2. The drug delivery device 1 is implemented as an injection device 30. The injection device 30 is a handheld pen-type injector. The injection device 30 may be implemented as a disposable injection device 30. It may comprise a pre-filled medicament container 6, for example, implemented as a cartridge or capsule. The medicament container 6 is arranged inside a cartridge holder 14. With a disposable injection device 30, the cartridge holder 14 may be non-detachably connected to a body 10 of a housing 32 of the injection device 30.

With other examples, the injection device 30 is a re-usable injection device, wherein the cartridge holder 14 is detachably connected to the body 10 for replacing an empty medicament container 6. At or near a distal end of the housing 32, hence at the distal end of the cartridge holder 14, there is provided a socket 28 configured to mount or to engage with an injection needle 15. The socket 28 may be implemented as a threaded socket and the injection needle 15 may comprise a needle hub begin correspondingly threaded to provide a threaded engagement with the socket 28.

Typically, the injection needle 15 is protected by an inner needle cap 16 and either by an outer needle cap 17 and/or a protective cap 18 that is configured to enclose and to protect a distal section of the housing 32 of the injection device 30. The body 10 may comprise and form a main housing part configured to accommodate a drive mechanism 34 as shown in FIG. 2. The cartridge holder 14 may be regarded as a distal housing component of the injection device 30. The cartridge holder 14 may be permanently or releasably connected to the body 10 or main housing.

The medicament container 6 comprises a cylindrically-shaped or tubular-shaped barrel 25 sealed in proximal direction 3 by a stopper 7 located inside the barrel 25. The medicament container 6 may be prefilled with a liquid medicament 27. The stopper 7 is displaceable relative to the barrel 25 of the container 6 in a distal direction 2 by a piston rod 20 of the drive mechanism 34. A distal end of the medicament container 6 is sealed by a pierceable seal 26 configured as a septum and being pierceable by a proximally directed tipped end of the injection needle 15. By attaching the injection needle 15 to the distal end of the cartridge holder 14 the seal 26 of the medicament container 6 is penetrated thereby establishing a fluid transferring access to the interior of the medicament container 6.

When the injection device 1 is configured to administer, for example, human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise a sleeve shaped knob at the proximal end of the housing 32 of the injection device 30.

As shown further in FIGS. 1 and 2, the body 10 comprises a dosage window 13 that may be in the form of an aperture in the body 10. The dosage window 13 permits a user to view a limited portion of a number sleeve (not illustrated) that is configured to move when the dose dial 12 is turned. The number sleeve and the dosage window 13 provide a visual indication of a dose currently set. The dose dial 12 may be rotated on a helical path with respect to the body 10 when turned during setting and/or dispensing or expelling of a dose.

With some other type of injection device, the dose dial 12 may be locked in longitudinal direction to the body 10. It may be then limited to a rotational movement relative to the body 10 for setting of the dose.

The injection device 30 may be configured so that turning the dosage knob 12 causes a mechanical click sound to provide acoustical feedback to a user. When the needle 15 is stuck into a skin portion of a patient, and when the trigger 11 or injection button is pushed, the dose of the liquid medicament displayed in the dosage window 13 will be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dose dial 12.

In the illustrated embodiment, during delivery of the insulin dose, the dose dial 12 is turned to its initial position in an axial movement, that is to say without rotation, while the number sleeve is rotated to return to its initial position, e.g., to display a dose of zero units.

The injection device 30 may be used for several injection processes until either the medicament container 6 is empty or the expiration date of the medicament in the injection device 1 (e.g., 28 days after the first use) is reached.

At least some components of an example of a drive mechanism 34 are illustrated in more detail in FIG. 2. The drive mechanism 34 comprises numerous mechanically interacting components. A flange like support of the body 10 comprises a threaded axial through opening threadedly engaged with a thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the stopper 7 of the medicament container 6. During a dispensing action the piston rod 20 rotates relative to the body 10 thereby experiencing a distally directed advancing motion relative to the body 10 and hence relative to the barrel 25 of the container 6. As a consequence, the stopper 7 of the medicament container 6 is displaced in distal direction 2 by a well-defined distance due to the threaded engagement of the piston rod 20 with the body 10.

The body 10 is provided with the dosage window 13 through which a part of the outer surface of the number sleeve can be seen. The body 10 is further provided with a helical rib at an inside sidewall portion of an insert piece 62, which helical rib is to be seated in a helical groove of the number sleeve. The tubular shaped insert piece 62 is inserted into the proximal end of the tubular shaped body 10. Alternatively, such a helical rib may be also provided directly on an inside of the sidewall of the body 10. The helical rib as well as the insert piece 62 is rotationally and axially fixed to the body 10. There may be provided first and second stops on the body 10 to limit a dose setting procedure during which the number sleeve is rotated in a helical motion relative to the housing 32.

The dose dial 12 in form of a dose dial grip is disposed about an outer surface of the proximal end of the number sleeve. An outer diameter of the dose dial 12 typically corresponds to and matches with the outer diameter of a proximal end of the body 10. The dose dial 12 is secured to the number sleeve to prevent relative movement there between. The dose dial 12 is provided with a central opening.

A trigger 11, also denoted as dose button is substantially T-shaped. It is provided at a proximal end of the injection device 30. A stem of the trigger 11 extends through the opening in the dose dial 12. The stem and hence the trigger 11 is retained for limited axial movement relative to the number sleeve. A head of the trigger 11 is generally circular. The trigger side wall or skirt extends from a periphery of the head and is further adapted to be seated in a proximally accessible annular recess of the dose dial 12.

To dial a dose a user rotates the dose dial 12, along a dose incrementing direction 4, e.g., clockwise. Dialing of a dose may be accompanied by a clicking sound. In this way, audible and/or tactile feedback of the dose being dialed is provided. Dialing of a dose is further accompanied by a rotation of the number sleeve, which starts to extend from the body 10 towards the proximal direction 3 when dialed along a dose incrementing direction 4, e.g., in a clockwise sense.

The number sleeve, the dose dial 12 and the trigger may form part of a dial extension 70, hence and assembly of components of the drive mechanism 34 that starts to extend or to displace from the proximal end of the body 10 as a dose is dialed. During dispensing of a dose, hence when a user depresses the trigger 11 in distal direction 2, the dial extension 70 is subject to a distally directed movement relative to the body 10, hence along the distal direction 2. During such a dispensing motion, the number sleeve is subject to a rotation along a dose decrementing direction 5, e.g. counter-clockwise.

The expelling mechanism or drive mechanism 34 as described above is only exemplary for one of a plurality of differently configured drive mechanisms that are generally implementable in a disposable or re-usable pen-injector. The drive mechanism as described above is explained in more detail e.g. in WO2004/078239A1, WO 2004/078240A1 or WO 2004/078241A1 the entirety of which being incorporated herein by reference.

Generally, the stopper 7, typically made of an elastomeric material, such as natural or synthetic rubber is in fluid tight engagement with an inside surface of the barrel 25. Movement of the stopper 7 relative to the barrel 25 is therefore inevitably subject to attic answers or dynamic friction. To reduce the friction between the stopper 7 and to provide a rather smooth displacement of the stopper 7 relative to the barrel 25 an inside surface of the barrel 25 can be provided with a lubricant 102.

The injection device 30 as illustrated in FIGS. 1 and 2 is only one example of a drug delivery configured for use with a medicament container 6 configured to store a liquid medicament and being provided with a piston or stopper slidably displaceable along the sidewall of the barrel 25.

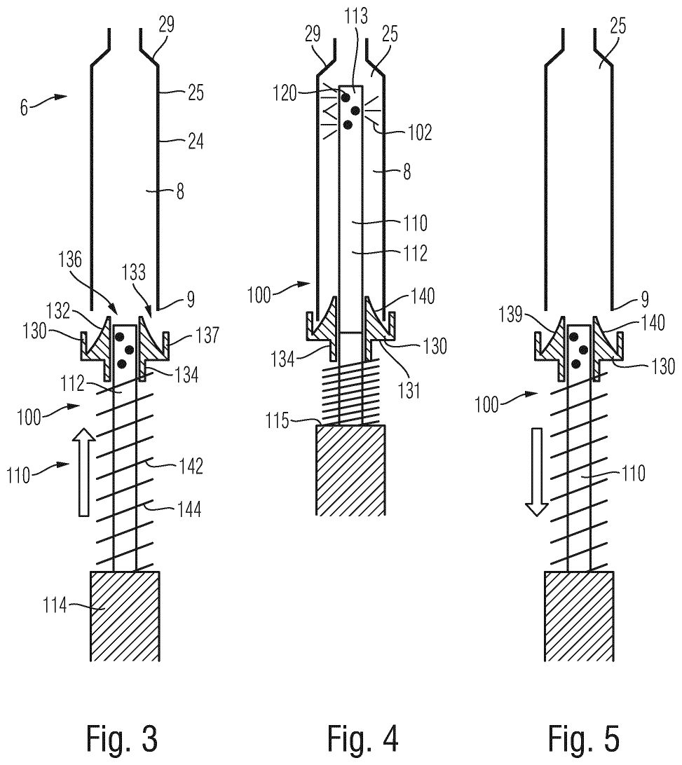

In FIGS. 3-5 one example of an applicator device 100 for depositing or applying a lubricant 102 onto an inside surface of the barrel 25 of the medicament container 6 is described. The applicator device 100 comprises a sprayer 110 defining a longitudinal direction and being sized for insertion into an interior 8 of the barrel 25 of the medicament container 6. The sprayer 110 is configured and sized to become inserted along the longitudinal direction of the sprayer 110, typically coinciding with the longitudinal direction of the barrel 25. The applicator device 100 further comprises a centering element 130. The centering element 130 is connected to the sprayer 110. The centering element 130 is operably and/or mechanically engageable with the barrel 25 in a predefined position or orientation.

The centering element 130 is configured to position, to orient and/or to align the sprayer 110 relative to the barrel 25. The barrel 25 may comprise a cylindrical sidewall 24. Towards a distal longitudinal end the sidewall 24 merges into a radially narrowing shoulder portion 29. Towards the opposite longitudinal end, hence towards the proximal end 9 the cylindrical barrel 25, i.e. the sidewall 24 is open to receive the sprayer 110 and/or the centering element 130.

With a cylindrically-shaped barrel 25 and with a cylindrical sidewall 24 the centering element 130 is configured to align, to position and/or to orient the sprayer 110 with regards to the transverse plane, i.e. perpendicular to the longitudinal direction of the sprayer 110 and/or perpendicular to the longitudinal direction of the barrel 25. In this way and when the sprayer 110 is inserted into the interior 8 of the barrel 25 the sprayer 110 can be kept at a predefined radial or transverse distance to the sidewall 24 of the barrel 25. Typically, the sprayer 110 is aligned and/or positioned in a radial or transverse central region of the barrel 25 by way of the centering element 130.

By aligning, orienting or positioning the sprayer 110 in a radial central region of the barrel 25 the radial or transverse distance between the sprayer 110 and the surrounding sidewall 24 of the cylindrical barrel 25 can be kept substantially constant as seen in circumferential direction. Along the tangential or circumferential direction the sprayer 110 can be kept or confined at a constant distance to the inside surface of the sidewall 24 of the barrel 25. In this way, a rather homogeneous deposition of the lubricant 102 onto the inside surface can be provided.

With some examples, presently not illustrated, the centering element 130 is shaped to enter the interior 8 of the barrel 25. Here, the centering element may radially or transversally protrude outwardly from the longitudinally shaped sprayer 110. It may serve as a radial or transverse spacer between the inside surface of the barrel 25 and the sprayer 110. Here, the centering element 130 and the sprayer 110 may be fixed relative to each other. Hence, they may not be allowed to move relative to each other. Insertion of the sprayer 110 into the interior 8 and removal of the sprayer 110 from the interior 8 may also come along with a respective sliding motion of the centering element 130 relative to the barrel 25 in longitudinal direction.

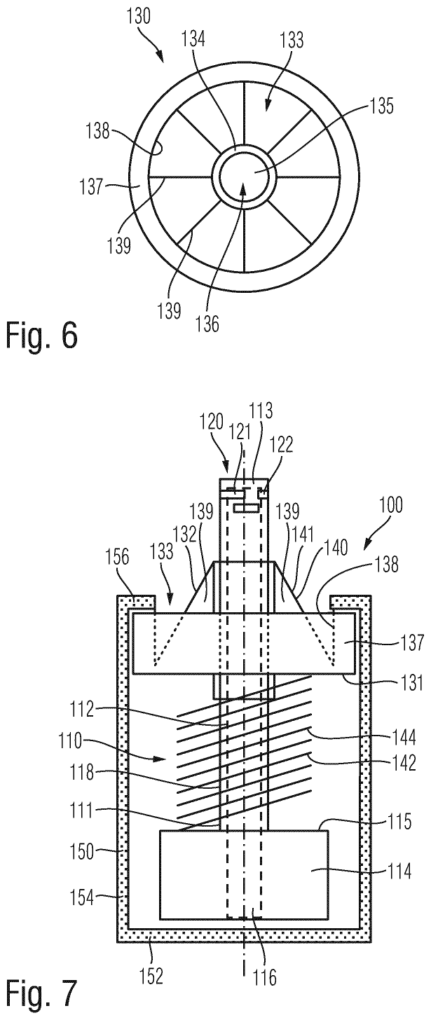

With the example as illustrated in FIGS. 3-7 the sprayer 110 is movable relative to the centering element 130 with regards to the longitudinal direction. Here, the applicator device 100 comprises a sliding guide 136 by way of which the sprayer 110 is slidably displaceable relative to the centering element 130 with regards to the longitudinal direction. As it becomes further apparent from the detailed illustration of the applicator device 100 of FIGS. 6 and 7 the centering element 130 comprises a central hub section 134 provided with a through opening 135 sized to receive the sprayer 110 therethrough. Here, the central hub section 134 comprises a circular-shaped through opening 135 that matches and correlates with the outer diameter or outer cross-section of the sprayer 110. In this way the sprayer 110, which comprises a longitudinal shaft section 112 is slidably guided in longitudinal direction in the through opening 135 of the central hub section 134. Here, the longitudinal shaft section 112 radially confined in the through opening 135 form or constitute the sliding guide 136.

The centering element 130 further comprises a distally facing abutment 132 by way of which the centering element 130 is axially or longitudinally engageable with the proximal end 9 of the barrel 25 of the medicament container 6. Such an abutment configuration is shown in FIG. 4. The radial or transverse extent of the centering element 130 is larger than the diameter or transverse cross-section of the barrel 25. Accordingly, the centering element 130 is blocked from entering the interior 8 of the barrel 25. As the applicator device 100 is moved in distal longitudinal direction relative to the barrel 25 the centering element 130 with its distally facing abutment 132 in longitudinal abutment with the proximal end 9 of the barrel 25 as illustrated in FIG. 4.

As the applicator device 100 is moved further in distal direction relative to the barrel 25 the sprayer 110 starts to move relative to the centering element 130 in distal direction and slides into the interior 8 of the barrel 25 until it reaches a final insert configuration as illustrated in FIG. 4. The final insert configuration may be characterized by the sprayer 110 reaching the shoulder portion 29 of the barrel 25 or reaching the distal end of the cylindrically-shaped sidewall 24 of the barrel 25.

A distal end 113 of the shaft section 112 of the sprayer 110 is provided with a nozzle 120. The nozzle 120 may be implemented as a spray nozzle configured and/or operable to generate an atomized spray of the lubricant 102 when a respective portion of the liquid lubricant 102 is forced through an inner bore 116 of the shaft section 112.

As further illustrated in FIGS. 3-5 and 7 the centering element 130 and the sprayer 110 are mechanically engaged through a restoring element 142, presently implemented as a spring 144, in particular as a compression spring. A distal end of the restoring element 142 is in longitudinal abutment with a proximally facing abutment 131 of the centering element 130. A proximal longitudinal end of the restoring element 142 is in longitudinal abutment with a distally facing portion of the sprayer 110. Here, the sprayer 110 comprises a base section 114 provided with a distally facing abutment 115 protruding radially outwardly from the cylindrically-shaped shaft section 112. The proximal end of the restoring element 142 is in longitudinal abutment with the distally facing abutment 115 of the sprayer 110.

In this way the sprayer 110 is slidably displaceable in longitudinal direction relative to the centering element 130 against the mechanical restoring force provided by the restoring element 142. When moving the sprayer 110 in longitudinal direction into the interior 8 of the barrel 25 towards the final insertion configuration as shown in FIG. 4 the restoring element 142 is biased. For removing of the sprayer 110 from the interior 8 of the barrel 25 the restoring element 142 provides a respective withdrawal force onto the sprayer 110, thereby increasing the distance between the abutment 115 of the sprayer 110 and the abutment 131 of the centering element 130.

The applicator device 100 as illustrated in FIG. 7 may further comprise a retainer 150, e.g. enclosing the sprayer 110 and the centering element 130. The retainer 150 comprises a retainer base 152 that may be in longitudinal abutment with the sprayer 110. The retainer base 152 may form a housing or a mechanical support for the base section 114 of the sprayer 110. The retainer base 152 may be open or may comprise at least one or several drainage holes to support drainage of excess lubricant. The retainer base 152 may comprise numerous strut or beams extending in radial direction providing axial support for the base section 114 of the sprayer 110.

The retainer 150 further comprises a retainer sidewall 154 extending in longitudinal direction. At a predefined longitudinal distance from the retainer base 152 the retainer 150 comprises a radially inwardly extending flange section 156 or at least two inwardly extending protrusions. The flange section 156 or protrusions is or are in longitudinal abutment with a distally facing side of the centering element 130. In this way, an uncontrolled detachment of the centering element 130 from the sprayer 110 can be effectively prevented.

Moreover, a relaxing of the restoring element 142 does not lead to an uncontrolled disassembly of the centering element 130 and the sprayer 110. In other words, the centering element 130 is movable in longitudinal direction relative to the sprayer 110 as well as relative to the retainer 150. A mechanical actuator for inducing a relative longitudinal movement between the centering element 130 and the sprayer 110 may be attached to the retainer 150. A distally directed displacement present to the retainer 150 may be transferred to the sprayer 110 through a longitudinal abutment of the retainer base 152 and the base section 114 of the sprayer 110 while the distally facing abutment 132 of the centering element 130 gets in mechanical, hence longitudinal engagement with the proximal end 9 of the barrel 25.

For a longitudinal abutment with the proximal end 9 of the barrel 25 the centering element 130 comprises at least one of the abutment 132 and a receptacle 133. The abutment 132 is configured to abut in longitudinal direction against the proximal end 9 of the barrel 25. The receptacle 133 is configured to receive the proximal end 9 of the barrel 25. As further indicated in FIGS. 3-5 and 7 the centering element 130 comprises a slanted section 140. The slanted section 140 may be formed by a cone shaped or inclined shaped structure of the abutment 132 and/or of the receptacle 133. The slanted section 140 provides a kind of a radial self-centering as the centering element 130 gets in longitudinal abutment with the circular shaped proximal end 9 of the barrel 25.

In the presently illustrated example the slanted section 140 connects the central hub section 134 of the centering element with an outer sidewall 137 of the centering element 130. The outer sidewall 137 comprises a diameter or cross-section being larger than a respective diameter or cross-section of the proximal end 9 of the barrel 25. The slanted section 140 is provided on numerous radially outwardly extending connecting wall sections 139 provided radially between the central hub section 134 and the outer sidewall 137. As it is particularly illustrated in FIG. 7 the slanted section 140 extends from an inside surface 138 of the outer sidewall 137 in longitudinal distal direction and radially inwardly towards the central part section 134. The radial extent of the connecting wall sections 139 is smallest at their distal ends and increases continuously and gradually towards their proximal ends.

As further illustrated in FIG. 7 with the dotted lines indicating the cross-section of the centering element 130, a proximal end of the connecting wall sections 139 terminates and/or adjoins with a proximal end portion of the outer sidewall 137. In this way at least a portion of the inside surface 138 of the outer sidewall 137 faces towards the slanted section 140 and forms a triangular shaped receptacle 133 for the proximal end 9 of the barrel 25.

The receptacle 133 is confined in radial direction by the inside surface 138 of the outer sidewall 137. Towards the proximal direction and the inside direction the receptacle 133 is confined by a distally facing edge 141 of the connecting wall sections 139. The radial or transverse dimension of the central hub section 134 and/or a radial or circumferential extent of the distal portion of the connecting wall sections 139 is smaller than a diameter or cross-section of the proximal end 9 of the barrel. In this way it can be provided that the centering element 130 is subject to a radial or transverse self-centering as it gets in longitudinal abutment with the proximal end 9 of the barrel 25.

In case of an initial radial or transverse misalignment between the barrel 25 and the centering element 130 one of the numerous connecting wall sections 139 engages with the proximal end 9 prior to other connecting wall sections 139. Due to the slanted shape of the distally facing edge 141 of the connecting wall sections 139 the centering element 130 will be subject to a respective movement in radial or transverse direction relative to the barrel 25 until at least three of the connecting wall sections 139 get in longitudinal abutment with the proximal end 9 of the barrel 25.

Typically, there are provided at least three connecting wall sections 139 distributed equidistantly along the outer circumference of the central hub section 134. The connecting wall sections 139 project radially outwardly from the central hub section 134.

The central hub section 134 and the outer sidewall 137 as well as the connecting wall sections 139 may be integrally or unitarily formed. They may be provided as a single piece.

With some examples, the centering element 130 may comprise or may be provided as an injection molded plastic component. Such an injection molded plastic component can reproduced rather cost efficient. Moreover, the plastic material of the centering element 130 is of particular benefit to avoid any damages to the barrel 25 and/or to reduce mechanical shock to the barrel 25 when the respective centering element 130 gets in axial or longitudinal abutment with the barrel 25.

With some examples the centering element comprises or is made of a metallic material, such as stainless steel. Use of a metallic material for the centering element 130 and/or for the shaft 112 or sprayer 110 is of particular benefit when using the applicator device in a sterile or aseptic environment. It may then easily withstand a sterilization procedure such as steam sterilization that takes place above 100° C., e.g. at about 123° C. For some applications where sterility is not required, plastic components may be used for the sprayer and/or for the centering element.

Generally and in order to obtain a radial or transverse self-centering of the centering element 130 and the barrel 25 it is sufficient when only one of the abutment 132 and the receptacle 133 comprises a slanted section 140. In the presently illustrated examples only the connecting wall sections 139 are provided with a slanted section 140 facing in distal direction. Alternatively, but not illustrated, it is also conceivable that the inside surface 138 of the outer sidewall 137 comprises a slanted section. Here, the inside surface 138 may be slanted or beveled as seen from the distal direction towards the proximal direction radially inwardly. With the presently illustrated example the distally facing edge 141 of the connecting wall section 139 will get in axial and/or radial abutment with an inside section of the proximal end 9 of the barrel 25.

When the slanted section 140 would be provided on the inside surface 138 of the outer sidewall 137 the slanted section 140 would engage with an outside portion of the proximal end 9 of the barrel 25.

Instead of the connecting wall sections 139 there may be provided a closed surface or closed portion extending between the central hub section 134 and the outer sidewall 137. Here, the central hub section 134 may be simply provided with a radially outwardly extending flange featuring a slanted section comparable to the distal edge 141 or the slanted section 140 of the connecting wall sections 139. In particular, a flange section extending radially outwardly from the central hub section 134 may comprise a cone-like shape. At or near a bottom of the central hub section there may be provided a through opening providing a kind of a drain hole allowing for a drainage of excess lubricant.

With the presently illustrated example, wherein the central hub section 134 is interconnected through numerous connecting wall sections 139 with the outer somewhat rim-shaped sidewall 137 it is of particular benefit that excess lubricant 1002 drains out of the interior 8 of the barrel 25. In the configuration as illustrated in FIG. 4 and when the proximal end 9 of the barrel 25 is oriented downwardly excess lubricant 102 may rinse down and drain along the inside surface of the barrel 25. It may leave the interior 8 of the barrel 25 unhinderedly.

In the present example the slanted section 140 of the connecting wall sections 139 is of rather straight shaped. With regard to the geometry of the connecting wall sections 139 numerous variations are generally conceivable. It is for instance conceivable that the slanted section 140 comprises a curved profile as seen in a plane defined by a longitudinal and radial direction coinciding with the plane of the connecting wall section 139. A curved profile may comprise a convex or concave shaped slope of the slanted section 140 and/or of the distally facing edge 141 of the connecting wall sections 139.

The centering element 130 contacting the barrel 25 is designed and configured that excess of lubricant may drain and not being held back in the centering element 130. As illustrated by the sequence of FIGS. 3 to 5, the sprayer 110 may dive into the medicament container 6 until it reaches a maximum insert configuration as illustrated in FIG. 4. During this insertion there may be no spraying of the lubricant. 102. The spray process may be started with or after a start of withdrawal of the sprayer 110 in proximal direction from the barrel 25. There may be a continued spraying provided by the sprayer 110 as the sprayer 110 is withdrawn from the medicament container 6.

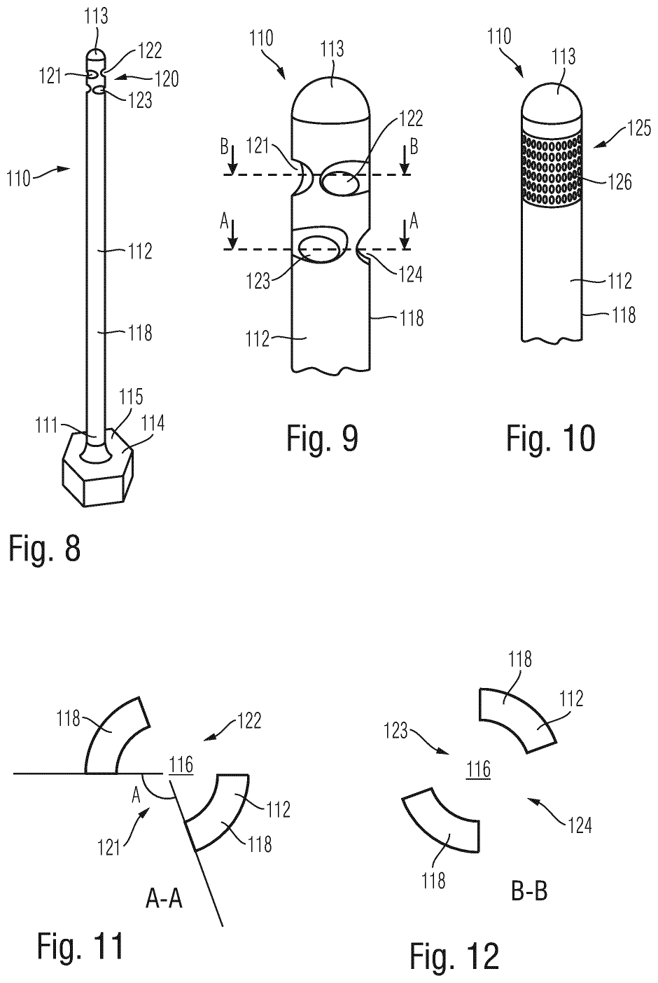

The sprayer 110 is illustrated in FIG. 8 in a separate perspective illustration. The sprayer 110 comprises an elongated shaft section 112. The shaft section 112 may be of cylindrical geometry. The shaft section 112 comprises a distal end 113. The distal end 113 is provided with a nozzle 120, typically implemented as a spray nozzle 120. The elongated shaft section 112 comprises an oppositely located proximal end 111. Beyond the proximal end 111 the shaft section 112 is connected to a radially widened base section 114. The base section 114 comprises a distally facing abutment face 115 protruding flange-like from the shaft section 112 and facing in distal direction. The abutment 115 provides support for the proximal end of the restoring element 142.

The shaft section 112 comprises an elongated or longitudinally extending sidewall 118. The shaft section 112 may be constituted by the longitudinally extending sidewall 118. It may substantially coincide with the sidewall 118. The shaft section 112 and hence the sidewall 118 is hollow. It comprises a longitudinally extending bore 116. The bore 116 extends from the open proximal end 111 of the shaft section 112 to the oppositely located distal end 113. The distal end 113 is closed. The nozzle 120 in fluid communication with the longitudinal bore 116 is located in the sidewall 118.

As illustrated in greater detail in FIGS. 9, 11 and 12 the nozzle 120 comprises numerous orifices 121, 122, 123, 124. The orifices 121, 122 are located on a common longitudinal level or position, e.g. at a first virtual transverse plane. The orifice is 123, 124 are also located on same longitudinal position. They may be provided on a second virtual transverse plane. The first and the second virtual transverse claims are longitudinally offset from each other. They may be oriented parallel to each other. The first orifice 121 and the second orifice 122 are arranged diametrically opposite to each other. Each orifice 121, 122 comprises a comparatively large opening angle. As illustrated in FIG. 11 the opening angle A of the orifice 121 is about 90° or even larger than 90°. The opening angles may range between 45° and 90° or between 45° and 135°. In this way, a rather widespread atomized spray can be applied to the inside surface of the barrel 25.

The third and the fourth orifices 123, 124 as illustrated in FIG. 12 may comprise a similar or identical geometry compared to the first and second orifices 121, 122. The orientation of the third and fourth orifices 123, 124 is circumferentially offset from the position or orientation of the first and second orifices 121, 122. The first and second orifices 121, 122 are longitudinally offset from the third and fourth orifices 123, 124.

In this way and in a projection in longitudinal direction as illustrated in the cross-sections of FIGS. 11 and 12 the first and second orifices 121, 122 may overlap in circumferential direction or with regard to a circumferential position with the portions of the sidewall 118 extending between the third and the fourth orifices 123, 124; and vice versa. In this way, the entire surrounding and circumference of the shaft section 112 can be homogeneously provided with the atomized spray of lubricant

With other, non-illustrated examples there may be only provided two or three orifices 121, 122, 123, wherein the two or three orifices are offset from each other both in longitudinal direction as well as in circumferential direction.

The numerous orifices 121, 122, 123, 124 provide a 360° spray profile to reach all areas of the container walls with the lubricant 102. Typically, numerous orifices are e.g. offset in longitudinal direction. Here, hence the orifice 121 on the one hand side and the orifices 123 and 124 on the other hand side may overlap circumferentially as seen in the longitudinal projection of FIGS. 11 and 12. In the same way the second orifice 121 on the one hand may overlap with at least a portion of at least one of the third orifice 123 and the fourth orifice 124.

In FIG. 10, a further example of a nozzle 125 at or near the distal end 113 of the shaft section 112 is illustrated. Here, the nozzle 125 comprises a nozzle grid 126. Typically, and with almost all embodiments the sprayer 110 is made of a metal or comprises a metal. Use of a metal material for realizing the nozzle 120, 125 is of particular benefit to provide a mechanically stable structure with comparatively small orifices 121, 122, 123, 124 configured and operable to generate a widespread atomized spray of lubricant.

The nozzle 125 and the nozzle grid 126 may comprise a sintered metal. They may be manufactured as a sintered sieve or filter.

With a further non-illustrated example the shaft section 112 and/or the entire sprayer 110 may be subject to a rotation relative to the barrel 25 with regard to its longitudinal axis while the lubricant 102 is injected or dispensed. Here, the number of orifices 121, 122, 123, 124 of a nozzle 120 and/or the circumferential diverging spray profile provided by the nozzle 120 may be reduced. Instead, the nozzle 120 is subject to a rotating or oscillating motion in circumferential or tangential direction during a spray process. For this, the shaft 112 may be rotationally supported on the base section 114. Alternatively, the base section 114 may be subject to a rotation, e.g. relative to the retainer 150 and social relative to the barrel 25 of the medicament container 6.

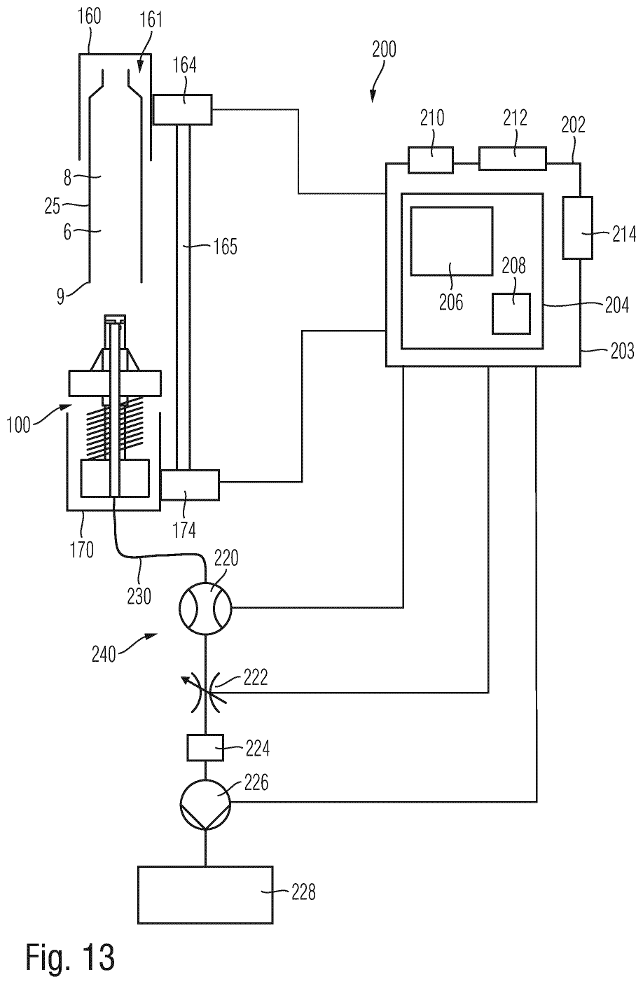

In the block diagram of FIG. 13 an applicator system 200 for depositing a lubricant 102 onto an inside surface of a barrel 25 of a medicament container 6 is illustrated. The applicator system 200 comprises an applicator device 100 as described above. Here, the medicament container 6 is fixed to a mount 160. The applicator device 100 is fixed to a further mount 170. The mount 160 and the mount 170 are displaceably arranged relative to each other, typically along the longitudinal direction as defined by the barrel 25 of the medicament container 6. For this at least one of the mount 160 and the mount 170 is provided with an electromechanical actuator 164, 174. The mounts 160, 170 are movable relative to each other along a guiding 165. For example, the mount 160 may move along the guiding 165 through the action of the actuator 164. Alternatively, the mount 170 may be subject to a movement along the guiding 165 by the actuator 174.

Generally, it is sufficient, when only one of the mounts 160, 170 is movable by an actuator 164, 174. The mount 160 and the mount 170 may be both mechanically engaged or mechanically connected to a longitudinally extending guiding 165. The guiding 165 may be also provided by a robotic device, e.g. by a robotic arm.

In the example of FIG. 13 the mount 160 is provided with a single receptacle 161 to receive a medicament container 6. Also the mount 170 is provided with a single receptacle 171 to receive a respective applicator device 100.

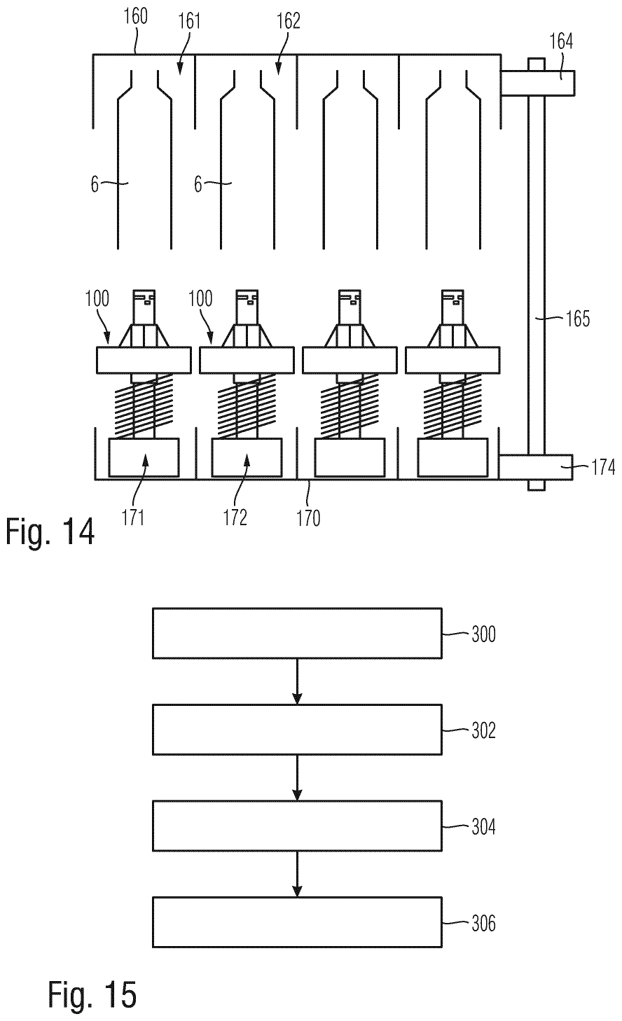

In the further example as illustrated in FIG. 14, the mount 160 is provided with numerous receptacles 161, 162 each of which configured to receive a medicament container 6. Correspondingly, the mount 170 comprises numerous receptacles 171, 172 each of which provided with an individual applicator device 100. The mount 160 may be provided with a row or with a two-dimensional array of medicament containers 6. Accordingly, also the mount 170 may be provided with a respective row or with an array of applicator devices 100. In this way, a large number of medicament containers 6 can be provided with the lubricant 102 simultaneously and in a single process step. Since each applicator device 100 is provided with a centering element 130 effective to provide a self-centering of the respective sprayer 110 with regard to the respective barrel 25 of the medicament container 6 a reliable and precise spray coating of the inside surface of the barrel 25 of the medicament containers can be provided at once.

In FIG. 15 numerous steps of a method of depositing a lubricant 102 on the inside surface of the barrel 25 of the medicament container 6 is illustrated. In a first step 300 at least one medicament container 6 is provided. In a subsequent step 302 a sprayer 110 of an applicator device 100 is inserted into the interior 8 of the medicament container 6. In step 304, the centering element 130 of the applicator device 100 is used to align, to orient and/or to position the sprayer 110 relative to the barrel 25 of the medicament container 6. Centering of the sprayer 110 typically occurs simultaneously or at the beginning of the insertion of the sprayer 110 into the interior 8. During or after insertion of the sprayer 110 into the interior 8 of the medicament container 6 a well-defined amount of the lubricant 102 is ejected from the sprayer 110 and is directed onto the inside surface of the barrel by using the sprayer 110.

Typically, injecting of the lubricant 102 from the sprayer 110 is conducted simultaneously with a movement of the sprayer 110 relative to the barrel 25, at least in longitudinal direction, optionally also with regards to the circumferential direction of the barrel 25. In this way, a rather homogeneous and precise spray coating of the inside surface of the barrel 25 of the medicament container 6 can be provided.

In further optional process steps, the medicament container 6 may be subject to a heat sterilization process, e.g. at temperatures of about 300° C. that may lead to a fixation of the lubricant on the respective surface of the medicament container 6. The lubricant 102 typically comprises a silicone oil or an emulsion containing a silicone-based lubricant, such as silicone oil. With some examples, the lubricant comprises or contains at least one of a Dimethylpolysiloxane, a fluorinated silicon oil and/or derivatives thereof.

Returning back to FIG. 13 the applicator system 200 comprises a controller 202 and a lubricant feeding system 240. The lubricant feeding system 240 comprises a pipe 230. The pipe 230 may comprise a flexible hose in flow connection with the inner bore 116 of the sprayer 110. The lubricant feeding system 240 further comprises a flow meter 220 and a flow regulator 222 as well as a bubble remover 224 in flow connection with the pipe 230. Furthermore, the lubricant feeding system 240 comprises a pump 226 and a lubricant reservoir 228. The pump 226, the flow regulator 222 as well as the flow meter 220 are connected to the controller 202. The controller 202 may be also connected to at least one of the electromechanical actuators 164, 174. The controller 202 typically comprises a housing 203 as well as an electronic circuit 204. The electronic circuit 204 typically comprises a processor 206 and a digital memory 208. The controller 202 is further provided with an input 210 and an output 212. The input 210 may comprise numerous actuation- or control elements, such as buttons or dials. The output 212 may comprise at least one of a visual display and a speaker. Optionally, the controller 202 is provided with a communication interface 214 providing a wired or wireless communication link with an external electronic device. The communication interface 214 may comprise a remote control of the entire applicator system 200.

The controller 202 is particularly configured to control both, a displacement of the medicament container 6 or medicament containers 6 relative to the applicator device 100 or applicator devices 100 as well as to start and to stop, and/or to control a spray delivery of the lubricant by the sprayer 110.

The controller 202 may keep track of and may control relative movement of the sprayer 110 and the barrel 25 of the medicament container 6. Typically, and when a final insertion position or configuration of the sprayer 110 inside the interior 8 of the barrel 25 has been reached the controller 202 may activate the pump 226 or may regulate the feeding of the lubricant 102 through and/or via the flow regulator 222. The flow of the lubricant 102 through the pipe 230 may be further controlled by the flow meter 220. In this way, the controller 202 is provided with a feedback regarding the amount of the lubricant 102 currently injected or dispensed.

The bubble remover 224 may be based on different air bubble removing concepts. The bubble remover 224 may comprise a semipermeable membrane by way of which optional bubbles contained in the lubricant 102 can be separated from the flow of the liquid lubricant. With some examples, the emulsion or the lubricant containing air or gas bubbles can be pushed against a microporous semipermeable membrane, e.g. a membrane made of Polytetrafluoroethylene. Through such a membrane the air or gas bubbles can be separated from the liquid lubricant.

Moreover but not illustrated the applicator system 200 may be further equipped with a control device configured and operable to control application of the lubricant on the barrel 25 of the medicament container(s) 6. Such a control device may be implemented as a visual control device, e.g. as an optical control device. It may be based on a Schlieren-optic method and may provide an in-process control to automatically detect medicament containers 6 that may not have received the correct amount of the applicant or that may exhibit an inhomogeneous lubricant distribution on the inside surface.

### LIST OF REFERENCE NUMBERS

1 drug delivery device
2 distal direction
3 first direction
4 second direction
5 dose decrementing direction
6 container
7 stopper
8 interior
9 proximal end
10 body
11 trigger
12 dose dial
13 dosage window
14 cartridge holder
15 injection needle
16 inner needle cap
17 outer needle cap
18 protective cap
19 protrusion
20 piston rod
21 bearing
22 threaded section
23 pressure foot
24 sidewall
25 barrel
26 seal
27 medicament
28 socket
29 shoulder portion
30 injection device
32 housing
34 drive mechanism
62 insert piece
70 dial extension
100 applicator device
102 lubricant
110 sprayer
111 proximal end
112 shaft section
113 distal end
114 base section
115 abutment
116 bore
118 sidewall
120 nozzle
121 orifice
122 orifice
123 orifice
124 orifice
125 nozzle
126 nozzle grid
130 centering element
131 abutment
132 abutment
133 receptacle
134 hub section
135 through opening
136 sliding guide
137 sidewall
138 inside surface
139 connecting wall section
140 slanted section
141 edge
142 restoring element
144 spring
150 retainer
152 retainer base
154 retainer sidewall
156 flange section
160 mount
161 receptacle
162 receptacle
164 actuator
165 guiding
170 mount
171 receptacle
172 receptacle
174 actuator
200 applicator system
202 controller
203 housing
204 electronic circuit
206 processor
208 memory
210 input
212 output
214 communication interface
220 flow meter
222 flow regulator
224 bubble remover
226 pump
228 reservoir
230 pipe
240 lubricant feeding system

1-15. (canceled)

16. An applicator device for depositing a lubricant onto an inside surface of a barrel of a medicament container, the applicator device comprising:
a sprayer defining a longitudinal axis, the sprayer being sized for insertion into an interior of the barrel of the medicament container, the sprayer being operable to eject the lubricant,
a centering element connected to the sprayer and operably engageable with the barrel in a predefined position or orientation to align the sprayer relative to the barrel.

17. The applicator device of claim 16, wherein the sprayer is movable relative to the centering element along the longitudinal axis.

18. The applicator device of claim 16, comprising a sliding guide through which the sprayer is slidably displaceable relative to the centering element.

19. The applicator device of claim 16, wherein the centering element comprises a central hub section with a through opening, wherein the sprayer comprises an elongated shaft section that is slidably supported in the through opening.

20. The applicator device of claim 19, wherein the centering element comprises an outer sidewall coaxial with the central hub section and enclosing the central hub section.

21. The applicator device of claim 20, wherein the central hub section and the outer sidewall are interconnected by one or more wall sections extending radially between the central hub section and the outer sidewall.

22. The applicator device of claim 16, comprising a restoring element engaged with the sprayer and engaged with the centering element;
wherein the sprayer is movable in a distal direction along the longitudinal axis and relative to the centering element against a restoring force of the restoring element.

23. The applicator device of claim 16, wherein the centering element comprises one or more of an abutment to abut relative to the longitudinal axis against a proximal end of the barrel, and a receptacle to receive the proximal end of the barrel.

24. The applicator device of claim 23, wherein the one or more of the abutment and the receptacle comprises a slanted section extending at a predefined angle relative to the longitudinal axis, wherein the slanted section is configured to induce a lateral movement of the centering element relative to the medicament container when the proximal end of the container engages with the one or more of the abutment and the receptacle.

25. The applicator device of claim 16, wherein the sprayer comprises:
a longitudinal fluid guiding bore enclosed by a longitudinally extending sidewall, and
a nozzle at or near a distal end of the longitudinally extending sidewall, wherein the nozzle is in fluid communication with the fluid guiding bore and wherein the nozzle comprises an orifice extending radially through the sidewall.

26. The applicator device of claim 25, wherein the nozzle comprises a second orifice, wherein the orifice is located circumferentially offset from the second orifice.

27. The applicator device of claim 26, wherein the nozzle comprises a third orifice, wherein the orifice is located circumferentially and axially offset from the third orifice.

28. The applicator device of claim 25, wherein the nozzle comprises a nozzle grid.

29. An applicator system for depositing a lubricant onto an inside surface of a barrel of a medicament container, the applicator system comprising:
an applicator device comprising a sprayer,
a lubricant feeding system in flow connection with the sprayer,
an electromechanical actuator operable to move the applicator device relative to a medicament container,
a controller connected to the lubricant feeding system and connected to the electromechanical actuator, the controller being operable to control a relative movement of the applicator device relative to the medicament container and to control ejection of a lubricant.

30. The applicator system of claim 29, wherein the sprayer defines a longitudinal axis, the sprayer being sized for insertion into an interior of the barrel of the medicament container, the sprayer being operable to eject the lubricant; and wherein the applicator device comprises a centering element connected to the sprayer and operably engageable with the barrel in a predefined position or orientation to align the sprayer relative to the barrel.

31. A method of depositing a lubricant on an inside surface of a barrel of a medicament container, the method comprising:
providing a medicament container comprising a barrel,
inserting a sprayer of an applicator device into the medicament container,
using a centering element of the applicator device to align and/or to center the sprayer relative to the barrel of the medicament container, the centering element connected to the sprayer, and
ejecting an amount of lubricant onto an inside surface of the barrel by using the sprayer.
